(19) United States
(12) Patent Application Publication
(10) Pub. No.: US 2005/0249678 A1
(43) Pub. Date: Nov. 10, 2005

(54) Multilayer tooth whitening strips

(76) Inventors: Mahmoud Hassan, Somerset, NJ (US); Guofeng Xu, Princeton, NJ (US); David Viscio, Monmouth Jct, NJ (US); Abdul Gaffar, Princeton, NJ (US)

Correspondence Address: HARNESS, DICKEY, & PIERCE, P.L.C, 7700 BONHOMME, STE 400, ST. LOUIS, MO, 63105, US

(21) Appl. No.: 10/842,152

(22) Filed: May 10, 2004

(51) Int. Cl. A61K 7/16; A61K 7/20

(52) U.S. Cl. 424/53

(57) ABSTRACT

Tooth-whitening films comprising a hydratable polymer and a whitening agent are disclosed in which the films have a hydrophobic coating on one surface and a tooth-adherent coating on the opposing surface. Flavorings can also be included in the hydratable polymer portion of the film or in either the hydrophobic coating or the tooth-adherent coating. Tooth-whitening methods that use the films are also disclosed.

### FIELD

This application relates generally to dental whitening compositions and methods and, more particularly, to multilayer tooth-whitening films and to methods therefor.

### BACKGROUND

Tooth whitening methods currently available for use at home involve application of peroxide-containing dentifrice compositions to the teeth. Such compositions can be applied by brushing or as a component of a strip that is applied to the teeth. The strips can comprise a plastic film with a tooth-whitening composition applied to the surface (see, for example, U.S. Pat. Nos. 5,894,017, 5,891,453, 6,045,811 and 6,419,906). Nevertheless, there remains a continuing need for new systems for applying peroxide-based whitening products to the teeth.

### SUMMARY

Accordingly, the present inventors have succeeded in developing methods and compositions for whitening the teeth involving application of a tooth-whitening strip to the teeth. The tooth-whitening strip can comprise a film which can comprise a water soluble, hydratable polymer and a whitening agent. The film can be hydrated by saliva upon application to the teeth and the whitening agent becomes solubilized or generates a bleaching agent that becomes solubilized which then acts to whiten the teeth. The film can also have a thin hydrophobic layer or coating on one of its surfaces, which upon application to the teeth is on the surface opposite that applied to the teeth. In certain embodiments, the film can have a thin adhesive layer or coating on the surface in contact with the teeth to facilitate adhesion of the film to the teeth. In certain embodiments, a flavoring agent can also be included in either the film, in the hydrophobic coating or the adhesive coating to mask any taste of the whitening agent.

Thus, in various embodiments, the present invention can involve a coated tooth-whitening film comprising a hydratable polymer and a whitening agent. The film can have a hydrophobic coating or layer on one surface and a tooth-adherent coating or layer on the opposing surface. By hydrophobic with respect to a substance, it is meant that the substance is antagonistic to water. Such substances have at least a portion of the molecule that is non-polar and, in some instances, the substance is immiscible with water, i.e. insoluble in water. By hydratable polymer it is meant that the polymer is capable of combining with water in a reversible association.

In various embodiments, the coated-tooth-whitening films of present invention can comprise a hydratable polymer and a whitening agent as well as a hydrophobic coating or layer on at least one surface, in which the hydrophobic coating contains a flavoring agent.

The present invention, in various embodiments, can also involve tooth-whitening methods in which the coated tooth-whitening films are contacted with a tooth for an effective tooth-whitening period. The effective tooth-whitening period is intended to refer to a contact time period producing a detectable whitening of the tooth. Reference herein to “a tooth” is intended to include the singular (tooth) and the plural (teeth). By detectable whitening it is meant that the whitening of the tooth can be visually observed or measured by any of various instruments following a given application period or following successive application periods of the same duration.

In various embodiments of the present invention, the hydratable polymer can comprises a film-forming polymer such as, for example, a poly(ethylene oxide)polymer having a number average, viscosity average, weight average or Z-average molecular weight of at least about 10,000 and not more than 10,000,000. The whitening agent can be a substance selected from the group consisting of peroxides, metal chlorites, perborates, percarbonates, persulfates, perphosphates, persilicates, peroxyacids and combinations thereof. The hydrophobic coating or layer can comprise a polymer selected from the group consisting of ethyl cellulose, propyl cellulose, isopropyl cellulose, butyl cellulose, t-butyl cellulose, cellulose acetate, polyvinyl acetate, shellac, acrylate copolymers such as those copolymers available under the trademark Eudragit® from Röhm America, Inc. (Piscataway, N.J.), a subsidiary of Degussa-Hüls Corporation (Ridgefield Park, N.J.), and combinations thereof. The tooth-adherent coating or layer can comprise any of a number of polymers, such as polyvinyl pyrrolidones, polyvinyl alcohols, polyvinyl phosphonates, poly ethylene phosphonates, polyacrylic acids and salts thereof, cross-linked polyacrylic acids and salts thereof, such as those sold under the trademark Carbopol® (Noveon, Inc., Brussels, Belgium), poly methyl methacrylates, polybutene phosphonates, polymaleates, vinylcaprolactam/sodium acrylate polymers, polystyrenes, phosphonate styrene polymers, terpolymers of acrylomethyl propyl sulphonic acid/methylacrylate/styrene monomers, polyaspartic acid, poly(2-acrylamido-2methylpropane sulfonate), copolymers of vinyl acetate and crotonic acid monomers, silicone polymers, polyacrylamide, polysiloxanes, alkyl cellulose polymers, hydroxyalkyl cellulose polymers, carboxy methyl celluloses and salts thereof, sodium alginate, alginic acid, copolymers of methyl vinyl ether and maleic anhydride such as those sold under the trademark Gantrez® (GAF Corporation) or combinations thereof.

In various embodiments, a flavoring agent can also be incorporated into the film, the hydrophobic coating layer or the adhesive coating layer. Such flavoring agents can be an essential oil, extract or flavoring aldehyde, ketone, ester or alcohol. The flavor imparted by the flavoring agent can be spearmint, peppermint, wintergreen, sassafras, clove, sage, eucalyptus, marjoram, cinnamon, lemon, lime, grapefruit, orange, apple, pear, peach, strawberry, cherry, apricot, watermelon, banana, coffee, cocoa, menthol, carvone, anethole or combinations thereof.

### DETAILED DESCRIPTION

The present invention, in various embodiments, can involve methods and compositions for whitening the teeth involving application of a tooth-whitening strip to the teeth. The tooth-whitening strip can comprise a film which can comprise a hydratable polymer and a whitening agent.

In various embodiments, the films of the present invention can be made in a thickness from about 20 to about 2000 μm, from about 50 to about 1000 μm or from about 50 to about 500 μm. the dried film can contain the whitening agent which, in various embodiments, can be a solid whitening agent in the inactive state. Hydration of the film by saliva in the oral cavity solubilizes the soluble whitening agent or a bleaching compound generated from the whitening agent in the film. The solubilization activates the whitening activity and releases active agent to the tooth surfaces to which the film is applied.

The rate at which the whitening agent is solubilized and released can be controlled by varying the film thickness, polymer properties, as well as the whitening agent composition and its concentration. The concentration of the whitening agent can be from about 0.1% to about 30%, from about 0.5% to about 25%, from about 1% to about 20%, from about 2% to about 10% or from about 3%, about 3.5% or about 4% to about 7% or about 10% by weight. Unless otherwise indicated, references to percentages herein are intended to mean percentages by weight (w/w).

The whitening agent can be any of a variety of peroxide-based bleaching agents, which deliver a hydrogen peroxide ion or an organic peroxide ion. Such compound include, for example, hydrogen peroxide, organic peroxide compounds, inorganic hydrogen peroxide generating compounds, organic peroxide generating compounds and combinations thereof.

Organic peroxide compounds include, for example, urea hydrogen peroxide (carbamide peroxide), glyceryl hydrogen peroxide as well as groups of peroxides classified according to the number and kind of organic functional groups attached to the oxygen atoms, such as, for example, alkyl hydrogen peroxide (R—O—O—H), dialkyl hydrogen peroxide (R—O—O—R′) peroxy acids (RCO—O—O—H), peroxy esters (RCO—OOR′), and diacyl peroxides (R—CO—O—O—CO—R′). Among such peroxides used in dental whitening are the diacyl peroxide, benzoyl peroxide and the peroxy acid monoperoxyphthalate.

In various embodiments, the whitening agent can also be a metal chlorite such as, for example, sodium chlorite, potassium chlorite, lithium chlorite, calcium chlorite, barium chlorite, or magnesium chlorite.

In various embodiments, the whitening agent can also an inorganic hydrogen peroxide generating compound such as, for example, alkali metal and alkaline-earth persulfate, dipersulfate, percarbonate, perphosphate, perborate, and persilicate salts such as, for example, sodium persulfate, sodium dipersulfate, sodium percarbonate, sodium perphosphate, sodium perborate, sodium persilicate, potassium persulfate potassium dipersulfate, potassium percarbonate, potassium perphosphate, potassium perborate, potassium persilicate, lithium dipersulfate, lithium percarbonate, lithium perphosphate, lithium perborate, lithium persilicate, calcium persulfate, calcium dipersulfate, calcium percarbonate, calcium perphosphate, calcium perborate, calcium persilicate, barium persulfate, barium dipersulfate, barium percarbonate, barium perphosphate, barium perborate, barium persilicate, magnesium persulfate, magnesium dipersulfate, magnesium percarbonate, magnesium perphosphate, magnesium perborate, and magnesium persilicate salts as well as sodium peroxide, potassium peroxide, lithium peroxide, calcium peroxide, barium peroxide and magnesium peroxide and combinations of any of the above compounds.

The whitening agent can also be one or more enzymes that release a bleaching peroxide compound such as, for example, oxidoreductases, such as a laccase or a related enzyme, and/or an oxidase such as glucose oxidase and hexose oxidase and/or a peroxidase (see U.S. Pat. No. 5,989,526 which is incorporated in its entirety by reference).

Suitable whitening agent can also be polymer-peroxide complex compound such as, for example, the PVP-hydrogen peroxide complex Peroxydone™ (ISP, Wayne, N.J.).

In various embodiments, the hydratable polymer can be any of a number of water-soluble film-forming polymers that are compatible with the peroxide compound. In particular, such synthetic polymers can include, for example, poly(ethylene oxide). The poly (ethylene oxide) can be a homopolymer or a copolymer or a block copolymer. Other homopolymers or copolymers including block copolymers, can include propylene oxide polymers, ethylene glycol polymers, methoxy(ethylene glycol)polymers and the like. Such polymers can have ethylene oxide moieties, propylene oxide moieties, ethylene glycol moieties and/or methoxy(ethylene glycol) moieties in an amount of at least 1%, at least about 5%, at least about 10%, at least about 20%, at least about 30%, at least about 40%, at least about 50%, at least about 60%, at least about 70%, at least about 80%, at least about 90% at least 99% or about 100% by weight.

In various embodiments, the hydratable polymer, and in particular, the ethylene oxide homopolymers can be a high molecular weight polymer. As used herein, molecular weight is intended to mean molecular weight as defined by number average molecular weight, viscosity molecular weight, weight average molecular weight or Z-average molecular weight. Such molecular weight representations can be measured by standard methods known in the art. The molecular weight of the hydratable polymer can be from about 10,000 to about 10,000,000, from about 100,000 to about 1,500,000 or about 2,000,000. Such poly(ethylene oxide)polymers are commercially available such as, for example, those available under the trademark Polyox® from Dow Chemical Company (Midland, Mich.).

In various embodiments, films can contain ethylene oxide polymers of a single average molecular weight or of a mixture of polymers of different average molecular weights. The ethylene oxide polymer can comprise from about 1% (w/w) to about 99% (w/w) of the hydratable film; in certain embodiments, from about 1% (w/w) to about 20% (w/w); from about 1% (w/w) to about 30% (w/w) or from about 1% (w/w) to about 50% (w/w); and in certain embodiments, from about 50% (w/w) to about 95% (w/w), or from about 60% (w/w) to about 85% (w/w).

In various embodiments, the film can be a Polyox® film and the whitening agent incorporated into the film can be sodium percarbonate.

The film can also contain other substances such as one or more plasticizers, which allows the adjustment of the strength and flexibility of the film. Typically, the plasticizers reduce the stiffness of films. Plasticizer compounds can include glycols such as propylene glycol or a low molecular weight polymer, for example, a polyethylene glycol such as any of the Carbowaxes of molecular weight from about 200 to about 600 available from Dow Chemical Company (Midland, Mich.). Polyhydric alcohols such as glycerin or propylene glycol, sorbitol, xylitol, glycerol esters such as glycerol triacetate(triacetin), triethyl citrate and natural oils such as mineral oil, caster oil and vegetable oils can also be used. Such compound can comprise from about 1% to about 50%, from about 5% to about 30% or from about 10% to about 25% by weight of the film.

Bulking agents which can modify the properties of the films can also be included. Such bulking agents can include water insoluble inorganic materials which can be in the form of particles such as, for example, silicon dioxide (silica), titanium dioxide, mica and Timeron mica, tricalcium phosphate, dicalcium orthophosphate (calcium monohydrogen phosphate), calcium carbonate and clays. Water-insoluble organic bulking agents can include celluloses, polyethylenes, polypropylene and various starches from potato, corn, oat, rice, wheat or tapioca and modified food starches such as, for example, maltodextrins. The bulking agents can be present in an amount of from about 1% to about 50%, from about 5% to about 30% or from about 10% to about 25% by weight of the film.

The film can also have a thin hydrophobic layer or coating on one of its surfaces, in particular, the surface of the film on the opposite side of the surface that contacts the teeth. The hydrophobic coating can provide a diffusion barrier to prevent the leakage of active whitening agent into undesired areas and to concentrate the whitening agent onto the teeth surface so as to increase the tooth-whitening effect.

The hydrophobic coating can be of a thickness of from about 10 nanometers to about 100 microns, from about 10 nanometers to about 10 microns, from about 10 nanometers to about 1 micron, from about 10 nanometers to about 500 nanometers, from about 50 to about 200 nanometers or from about 75 to about 100 nanometers or any value within the aforementioned ranges.

The hydrophobic coating can be comprised predominantly by hydrophobic molecules. Such hydrophobic molecules can have a hydrophobic component, i.e. a portion of the molecule that is non-polar and antagonistic to water. Nevertheless, in certain instances, such hydrophobic molecules can be miscible with water such as can be the case with surfactants. Hydrophobic molecules which are surfactants can have a hydrophilic portion which is contiguous with the hydratable film and a hydrophobic portion which directed away from the film. In various embodiments, the hydrophobic substances that can comprise the hydrophobic coating can be substance that are immiscible with water, i.e. insoluble in water, having an aqueous solubility of less than about 1% (w/w), less than about 0.01% (w/w) or less than about 0.001% (w/w). Such hydrophobic substances can be comprised by one or more hydrophobic polymers such as, for example, shellac, Eudragit®, polymethyl methacrylate and its copolymers, polyethylene, polypropylene, polyvinyl alcohol, polyesters, ethyl cellulose, propyl cellulose, isopropyl cellulose, butyl cellulose, t-butyl cellulose, cellulose acetate, and derivatives of polyvinyl alcohol such as, for example, polyvinyl acetate or silicone polymers.

The hydrophobic coating can also, in various embodiments, contain substances which are hydrophilic such as plasticizers as described above, so long as the coating as a whole provides a diffusion barrier to prevent the leakage of active whitening agent into undesired areas. The hydrophobic molecules can be present in the hydrophobic coating in an amount of from about 1% (w/w) to about 95% (w/w) and the plasticizers can be present in an amount of from about 1% (w/w) to about 50% (w/w).

The film can also have a thin adhesive layer or coating on the surface that contacts the teeth. In various embodiments, this coating is not necessarily adhesive in the dry state, but upon contacting the tooth surface it can become adhesively activated by an aqueous liquid such as water or saliva such that it adheres to the teeth. The adhesive coating can be comprised of peroxide-compatible polymers including polyvinyl pyrrolidone, polyvinyl alcohol, polyvinyl phosphonate, poly ethylene phosphonate, polyacrylic acids, poly methyl methacrylate, polybutene phosphonate, polymaleate, vinylcaprolactam/sodium acrylate polymers, polystyrene, phosphonate styrene, terpolymers of acrylomethyl propyl sulphonic acid/methylacrylate/styrene monomers (see U.S. Pat. No. 5,800,803), polyaspartic acid, poly(2-acrylamido-2methylpropane sulfonate), copolymers of vinyl acetate and a crotonic acid, silicone polymers, polyacrylamide, polysiloxanes (see U.S. Pat. Nos. 5,876,208; 5,888,491; 5,866,630; 5,607,663; 5,662,887), and alkyl or hydroxyalkyl substituted cellulose polymers such as, for example, methyl cellulose, ethyl cellulose, or propyl cellulose or hydroxymethyl cellulose, hydroxyethyl cellulose, hydroxypropyl cellulose, carboxy methyl cellulose (CMC), sodium alginate, alginic acid, copolymers of methyl vinyl ether and maleic anhydride or its corresponding acid such as those sold under the trademark Gantrez® (ISP, Wayne, N.J.) or combinations of any of the above. In various embodiments, the adhesive layer can also contain such additional substances as plasticizers as described above.

The structure of the multilayer film with hydrophobic coating and adhesive coating is such that the middle, core film contains the active whitening agent, such as sodium percarbonate in a Polyox® matrix with the adhesive coating and hydrophobic coating on opposing surfaces of the core film.

In certain embodiments, a flavoring agent can also be included in either the film, in the hydrophobic coating or the adhesive coating to mask any taste of the whitening agent. In various embodiments, the flavoring agent can be incorporated into the film, the hydrophobic coating layer or the adhesive coating layer. In various embodiments the flavoring agent can be incorporated into the adhesive layer which can be maintained in a dry condition prior to application to the teeth. This can decrease the likelihood of degradation of any flavoring agent that would otherwise be susceptible to degradation by the peroxide compound. The flavoring agents of the present invention can include any of those known to the skilled artisan, such as natural and artificial flavors. These flavoring agents can be synthetic flavor oils and flavoring aromatics, and/or oils, oleo resins and extracts derived from plants, leaves, flowers, fruits and so forth, and combinations thereof. Representative flavor oils include: spearmint oil, cinnamon oil, peppermint oil, clove oil, bay oil, thyme oil, cedar leaf oil, oil of nutmeg, oil of sage, and oil of bitter almonds. Also useful are artificial, natural or synthetic fruit flavors such as vanilla, chocolate, coffee, cocoa and citrus oil, including lemon, orange, grape, lime and grapefruit and fruit essences including apple, pear, peach, strawberry, raspberry, cherry, plum, pineapple, apricot and so forth. These flavorings can be used individually or in combination. Commonly used flavors include mints such as peppermint, wintergreen, spearmint, birch, anise and such fruit flavors, as cherry, lemon-lime, orange, grape, artificial vanilla, cinnamon derivatives, and others, whether employed individually or in combination. Flavorings such as aldehydes and esters including cinnamyl acetate, cinnamaldehyde, citral, diethylacetal, dihydrocarvyl acetate, eugenyl formate, p-methylanisole, and so forth can also be used. Further examples of aldehyde flavorings include, but are not limited to acetaldehyde (apple); benzaldehyde (cherry, almond); cinnamic aldehyde (cinnamon); citral, i.e., alpha citral (lemon, lime); neral, i.e. beta citral (lemon, lime); decanal (orange, lemon); ethyl vanillin (vanilla, cream); heliotropine, i.e., piperonal (vanilla, cream); vanillin (vanilla, cream); alpha-amyl cinnamaldehyde (spicy fruity flavors); butyraldehyde (butter, cheese); valeraldehyde (butter, cheese); citronellal (modifies, many types); decanal (citrus fruits); aldehyde C-8 (citrus fruits); aldehyde C-9 (citrus fruits); aldehyde C-12 (citrus fruits); 2-ethyl butyraldehyde (berry fruits); hexenal, i.e. trans-2 (berry fruits); tolyl aldehyde (cherry, almond); veratraldehyde (vanilla); 2,6-dimethyl-5-heptenal, i.e. melonal (melon); 2-6-dimethyloctanal (green fruit); and 2-dodecenal (citrus, mandarin); cherry; grape; mixtures thereof; and the like. Generally the flavoring can be incorporated in the film of the present invention in an amount ranging from about 1.0% to about 2.0% to about 10% by weight or from about 5% to about 8% by weight.

The poly(ethylene oxide) films of the present invention can be prepared using conventional extrusion or solvent casting processes. For example, to prepare a film by solvent casting poly(ethylene)oxide, the ethylene oxide polymers can be dissolved in a sufficient amount of a solvent which is compatible with the polymer. Examples of suitable solvents include water, alcohols, acetone, ethyl acetate or mixtures thereof. After a solution has been formed, a plasticizer can be added with stirring, and heat can be applied if necessary to aid dissolution, until a clear and homogeneous solution has been formed, followed by the addition of the whitening agent and any other ingredients such as flavors. The solution can be coated onto a suitable carrier material and dried to form a film. The carrier material can have a surface tension which allows the polymer solution to spread evenly across the intended carrier width without soaking in to form a destructive bond between the two substrates. Examples of suitable carrier materials include glass, stainless steel, Teflon® (DuPont, Wilmington, Del.), polyethylene-impregnated Kraft paper or polyester plastic liners.

Drying of the film can be carried out in a moderate to high-temperature air-bath using a drying oven, drying tunnel, vacuum drier, or any other suitable drying equipment, which does not adversely affect the active ingredient(s) or flavor of the film.

For ease of use, the dry film can be cut into pieces of suitable size and shape and packed into a suitable container.

To use the whitening film strip of the present invention, the film when applied to the teeth surface when hydrated by saliva in the oral cavity or pre-wetted by dipping the strip in water will adhere to the teeth in an appropriate manner. In this regard, the whitening strip can be formed to have a width dimension suitable to cover a row of teeth (upper or lower). Therefore, the whitening strip can be applied to the upper set of teeth, or to the lower set of teeth either separately or simultaneously. The length dimension of the whitening strip is determined by the amount of coverage desired. In this regard, the number of teeth which it is desired to whiten will determine the dimensions of the whitening strip. For instance, it may be desired to only whiten the front teeth, which are most easily seen by others. Accordingly, the length of whitening strip can be reduced in this case, as compared to the case where it is desired to whiten all of the teeth. The duration of application of whitening strip to the teeth will depend upon the type and concentration of the whitening agent, as well as the type and intensity of stain. Typically, the duration of application, also referenced herein as the tooth-whitening period, can be from about 1 minute to about 60 minutes or greater, from about 2 minutes to about 30 minutes or from about 5 minutes to about 10 minutes or any time period encompassed by the aforementioned ranges.

The present invention can be illustrated by the following examples.

### EXAMPLE 1

This example illustrates the preparation and testing of hydratable poly(ethylene oxide) films containing sodium percarbonate and having a hydrophobic coating.

Ten grams (g.) of polyethylene oxide (M.W. 200,000, Aldrich) was slowly added into about 85.0 g., de-ionized water heated to about 80° C., with vigorous stirring to form a translucent and viscous solution. To this solution, 2.5 g. glycerin was added and stirred for about 5 to 30 minutes. The mixture was cooled down to below 40° C. and 2.4 g. sodium percarbonate powder was added and thoroughly mixed. The resulting white mixture was cast onto a glass plate and allowed to dry overnight. A white dry film was formed which was readily peeled off from the glass plate. The dry film had a thickness of about 160 micrometers and contained an equivalent of about 3.4% hydrogen peroxide by weight of the film.

The whitening efficacy of this film (designated “Film A”) was examined by wetting and placing piece of the film (14 mm.times.15 mm, weighing 23 milligrams (mg.) against the surface of a stained bovine enamel slab prepared as described in “In Vitro Removal of Stain with Dentifrice”, G. K. Stookey, T. A. Barkhard and B. R. Schemerhorn, J. Dental Res., 61, 1236-9 (1982) and obtained from Oral Health Research Institute of Indiana University. A piece of wet paper towel was placed on top of the film to maintain the moisture. After 30 minutes, both the towel and the residue film were removed and the tooth was washed with water. The shade of the tooth was measured and recorded both before and after the treatment with a Minolta Chromometer Model CR 321 in which “L” is a measure of response to the eye to lightness and darkness, the higher the L value the whiter teeth appear.

For purposes of comparison, a commercially available tooth whitening strip of the type disclosed in U.S. Pat. No. 5,894,017 containing 5.2% by weight hydrogen peroxide (designated “Film B”) was also cut into a similar dimension which weighed 40 mg was used to treat a similarly stained bovine enamel slab for 30 minutes following the procedure of Example 1. The change in L values of the treated Films A and B are recorded in Table I below.

TABLE I

| | Weight | H₂O₂ | | | |
|---|---|---|---|---|---|
| Film | (mg) | wt % | Initial L | End L | dL |
| A | 23 | 3.4 | 37.32 | 62.09 | 24.77 |
| B | 40 | 5.2 | 37.43 | 44.67 | 7.24 |

The results recorded in Table I above show that the whitening efficacy of Film A prepared in accordance with the present invention is unexpectedly much higher than that of commercial white strip (Film B), despite having a significantly lower peroxide content

### EXAMPLE 2

This example illustrates the preparation and whitening of films with varying peroxide concentrations.

Series of films were prepared using 1.0 g poly(ethylene)oxide (M.W. approximately 400,000) which was slowly added with vigorous stirring into about 80.0 g hot de-ionized water heated to about 80° C. While maintaining the same temperature and continuing agitation, another 10.0 g poly(ethylene)oxide (M. W. approximately 200,000) was slowly added into the solution. After the complete dissolution of the polyethylene oxide, a translucent and viscous solution/suspension was formed. The solution was concentrated by heating and stirring at about 80° C. for another two hours. The solution was then cooled to room temperature (about 23° C.).

To the above solution, 3.3 g. glycerin was added and stirred for about 30 minutes, until a homogeneous solution was obtained. Subsequently, 3.0 g sodium percarbonate powder was added and thoroughly mixed with the poly(ethylene)oxide solution to form a white, thick gel-like suspension.

A free-standing film was prepared by casting the above gel-like suspension onto glass plates and drying at room temperature (about 23° C.) overnight. After drying, a film was formed and could be readily peeled off from the glass plates. The film was flexible and strong and could be bent or folded without breaking. Both thin and thick films were prepared. A thin film designated “C” had a thickness of about 100 um and contained about 2.9% hydrogen peroxide by weight.

A thicker film designated “Film D” had a thickness of 500 um and a hydrogen peroxide content of 3.4% by weight.

A third supported film was prepared by casting the poly(ethylene)oxide suspension prepared as above onto a flexible cotton cloth and dried in air. The resulting strip (designated “Film E”) had a hydrogen peroxide content of 1.5% by weight by weight of the supported film.

The whitening test procedure of Example I using stained bovine enamel slabs was repeated to determine the tooth-whitening efficacy of Films C, D and E. The shade change of the tooth was recorded using the Minolta Chromometer Model CR 321 both before and after the treatment. As a comparison, a piece of a commercially available whitening strip (Film B of Example I) was also cut into a similar dimension and used to treat another bovine tooth for 30 minutes, the strip is designated as film F in Table II. The results of these tests are recorded in Table II.

TABLE II

| Film | H₂O₂ wt % | Initial L | End L | dL |
|---|---|---|---|---|
| C | 2.9 | 28.98 | 47.56 | 18.58 |
| D | 3.4 | 38.28 | 65.21 | 26.93 |
| E | 3.0 | 40.07 | 61.82 | 21.75 |
| F | 4.6 | 33.33 | 41.54 | 8.21 |

The shade of the stained bovine enamels slabs after the 30 minute exposure to the Films C, D and E of the present invention showed superior whitening efficacy as compared to the comparative commercial whitening strip Film B or F.

### EXAMPLE 3

This Example illustrates the preparation and testing of hydratable poly(ethylene oxide) films containing sodium percarbonate and having a hydrophobic coating and adhesive coating.

The coated film can be prepared as follows. The core layer was made by thermally extruding Polyox® (M.W.=200K; The Dow Chemical Company, Midland Mich.) with sodium percarbonate. The thickness of this particular film was about 100 μm and the concentration of the sodium percarbonate was 5% by weight. The adhesive layer was then produced by casting and drying a thin film of an ethanol solution of PVP onto the Polyox® layer. The hydrophobic layer was prepared by first coating the uncoated side of the Polyox® layer with a thin layer of PVP, ethanol solution; then, immediately before this layer is dry, a film of ethylcellulose ethanol solution was cast on and dried. A multilayer structure was thus obtained.

### EXAMPLE 4

This example illustrates the whitening activity of the multilayer film of Example 3 using an in vitro method.

A small piece of the multilayer film prepared according to the method in Example 3, was placed against a wet bovine enamel tooth slab that had been artificially stained as described in Stookey, et al, J. Dental Res., 61, 1236-9, 1982 and obtained from Oral Health Research Institute of Indiana University. Sufficient pressure was applied to the back of the film to insure an adhesive contact between the film and the tooth surface. The film was allowed to stay on the tooth surface for 30 minutes and then removed. The tooth surface was then thoroughly washed and dried.

The lightness of the tooth surface was measured using a Minolta Chromometer Model CR 321 both before and after the treatment. As a comparison, a piece of a commercially available whitening strip (Film B of Example 1) was also cut into a similar dimension and used to treat another bovine tooth for 30 minutes. The dL reading was 18.35 for the multilayer film of the present invention. This is a greater whitening effect than produced by the commercially available whitening product in examples 1 and 2 which gave a value of about 7 to 8 in the same test.

### EXAMPLE 5

This example illustrates the stability of the core Polyox® layer in comparison to a commercially available whitening strip.

This test was carried out by sealing the samples of core Polyox® layer and a commercially available whitening strip (Film B of Example 1) into glass jars and aging at 120° F. for two weeks. The commercially available strip was first taken out of its original packaging pouch.

The Polyox® film lost about 25% peroxide activity in comparison to a loss of nearly 60% peroxide activity for the commercially available strip. Thus the films of the present invention provide a much more stable product than the commercially available product.

All references cited in this specification are hereby incorporated by reference. Any discussion of references cited herein is intended merely to summarize the assertions made by their authors and no admission is made that any reference or portion thereof constitutes relevant prior art. Applicants reserve the right to challenge the accuracy and pertinency of the cited references.

The description of the invention is merely exemplary in nature and, thus, variations that do not depart from the gist of the invention are intended to be within the scope of the invention. Such variations are not to be regarded as a departure from the spirit and scope of the invention.

1. A coated tooth-whitening film comprising a hydratable, film-forming polymer and a whitening agent, said film having a hydrophobic coating on one surface and a tooth-adherent coating on opposing surface.

2. A film according to claim 1, wherein said hydratable polymer comprises a poly(ethylene oxide)polymer having a number average, viscosity average, weight average or Z-average molecular weight of at least about 10,000 and not more than 10,000,000.

3. A film according to claim 1, wherein the whitening agent is selected from the group consisting of peroxides, metal chlorites, PVP-hydrogen peroxide complex, perborates, percarbonates, persulfates, perphosphates, persilicates, peroxyacids and/or its salts and combinations thereof.

4. A film according to claim 1, wherein a hydrophobic coating comprises a polymer selected from the group consisting of an ethyl cellulose, a propyl cellulose, an isopropyl cellulose, a butyl cellulose, a t-butyl cellulose, a cellulose acetate, a polyvinyl acetate, a shellac, an acrylate copolymer and combinations thereof.

5. A film according to claim 1, wherein the tooth-adherent coating comprises a polymer selected from the group consisting of polyvinyl pyrrolidones, polyvinyl alcohols, polyvinyl phosphonates, poly ethylene phosphonates, polyacrylic acids, polyacrylate salts, polymethacrylic acid, polymethacrylate salt, poly methyl methacrylates, polybutene phosphonates, polymaleates, vinylcaprolactam/sodium acrylate polymers, polystyrenes, phosphonate styrene polymers, terpolymers of acrylomethyl propyl sulphonic acid/methylacrylate/styrene monomers, polyaspartic acid, poly(2-acrylamido-2methylpropane sulfonate), copolymers of vinyl acetate and crotonic acid monomers, silicone polymers, polyacrylamide, polysiloxanes, alkyl cellulose polymers, hydroxyalkyl cellulose polymers, carboxy methyl cellulose polymers, sodium alginates, alginic acids, poly methyl vinyl ether/maleic anhydride copolymers, cross-linked polyacrylic acids, cross-linked polyacrylate salts and combinations thereof.

6. A film according to claim 1, wherein any one or more of the film, hydrophobic coating or adhesive coating further comprises a flavoring agent comprising an essential oil, extract or flavoring aldehyde, ketone, ester or alcohol that imparts a flavor selected from the group consisting of spearmint, peppermint, wintergreen, sassafras, clove, sage, eucalyptus, marjoram, cinnamon, lemon, lime, grapefruit, orange, apple, pear, peach, strawberry, cherry, apricot, watermelon, banana, coffee, cocoa, menthol, carvone, anethole and combinations thereof.

7. A coated tooth-whitening film comprising a hydratable polymer and a whitening agent said film having on at least one surface, a hydrophobic coating containing a flavoring agent.

8. A film according to claim 7, wherein said hydratable polymer comprises a poly(ethylene oxide)polymer having a number average, viscosity average, weight average or Z-average molecular weight of at least about 10,000 and not more than 10,000,000.

9. A film according to claim 7, wherein the whitening agent is selected from the group consisting of peroxides, PVP-hydrogen peroxide complex, metal chlorites, perborates, percarbonates, persulfates, perphosphates, persilicates, peroxyacids and/or its salts and combinations thereof.

10. A film according to claim 7, wherein the hydrophobic coating comprises a polymer selected from the group consisting of an ethyl cellulose, a propyl cellulose, an isopropyl cellulose, a butyl cellulose, a t-butyl cellulose, a cellulose acetate, a polyvinyl acetate, a shellac, an acrylate copolymer and combinations thereof.

11. A film according to claim 7, wherein the flavoring agent comprises an essential oil, extract or flavoring aldehyde, ketone, ester or alcohol that imparts a flavor selected from the group consisting of spearmint, peppermint, wintergreen, sassafras, clove, sage, eucalyptus, marjoram, cinnamon, lemon, lime, grapefruit, orange, apple, pear, peach, strawberry, cherry, apricot, watermelon, banana, coffee, cocoa, menthol, carvone, anethole and combinations thereof.

12. A tooth-whitening method comprising providing a coated tooth-whitening film comprising a hydratable polymer and a whitening agent, said film having a hydrophobic coating on one surface and a tooth-adherent coating on opposing surface; and contacting a tooth with said coated film for an effective tooth-whitening period.

13. A method according to claim 12, wherein said hydratable polymer comprises a poly (ethylene oxide)polymer having a number average, viscosity average, weight average or Z-average molecular weight of at least about 10,000 and not more than 10,000,000.

14. A method according to claim 12, wherein the whitening agent is selected from the group consisting of peroxides, metal chlorites, perborates, percarbonates, persulfates, perphosphates, persilicates, peroxyacids, salts of peroxyacids and combinations thereof.

15. A method according to claim 12, wherein the hydrophobic coating comprises a polymer selected from the group consisting of an ethyl cellulose, propyl cellulose, isopropyl cellulose, butyl cellulose, t-butyl cellulose, cellulose acetate, polyvinyl acetate, shellac and combinations thereof.

16. A film according to claim 12, wherein the tooth-adherent coating comprises a polymer selected from the group consisting of polyvinyl pyrrolidones, polyvinyl alcohols, polyvinyl phosphonates, poly ethylene phosphonates, polyacrylic acids, polyacrylate salts, polymethacrylic acid, polymethacrylate salt, poly methyl methacrylates, polybutene phosphonates, polymaleates, vinylcaprolactam/sodium acrylate polymers, polystyrenes, phosphonate styrene polymers, terpolymers of acrylomethyl propyl sulphonic acid/methylacrylate/styrene monomers, polyaspartic acid, poly(2-acrylamido-2methylpropane sulfonate), copolymers of vinyl acetate and crotonic acid monomers, silicone polymers, polyacrylamide, polysiloxanes, alkyl cellulose polymers, hydroxyalkyl cellulose polymers, carboxy methyl cellulose polymers, sodium alginates, alginic acids, poly methyl vinyl ether/maleic anhydride copolymers, cross-linked polyacrylic acids, cross-linked polyacrylate salts and combinations thereof.

17. A method according to claim 12, wherein the flavoring agent comprises an essential oil, extract or flavoring aldehyde, ketone, ester or alcohol that imparts a flavor selected from the group consisting of spearmint, peppermint, wintergreen, sassafras, clove, sage, eucalyptus, marjoram, cinnamon, lemon, lime, grapefruit, orange, apple, pear, peach, strawberry, cherry, apricot, watermelon, banana, coffee, cocoa, menthol, carvone, anethole and combinations thereof.

18. A tooth-whitening method comprising providing a coated tooth-whitening film comprising a hydratable polymer and a whitening agent said film having on at least one surface, a hydrophobic coating containing a flavoring agent; and contacting a tooth with said film for an effective tooth-whitening period.

19. A method according to claim 18, wherein said hydratable polymer comprises a poly (ethylene oxide)polymer having a number average, viscosity average, weight average or Z-average molecular weight of at least about 10,000 and not more than 10,000,000.

20. A method according to claim 18, wherein the whitening agent is selected from the group consisting of peroxides, PVP-hydrogen peroxide complex, metal chlorites, perborates, percarbonates, persulfates, perphosphates, persilicates, peroxyacids, salts of peroxyacids and combinations thereof.

21. A method according to claim 18, wherein the hydrophobic coating comprises a polymer selected from the group consisting of an ethyl cellulose, a propyl cellulose, an isopropyl cellulose, a butyl cellulose, a t-butyl cellulose, a cellulose acetate, a polyvinyl acetate, a shellac, an acrylate copolymer and combinations thereof.

22. A method according to claim 18, wherein the flavoring agent comprises an essential oil, extract or flavoring aldehyde, ketone, ester or alcohol that imparts a flavor selected from the group consisting of spearmint, peppermint, wintergreen, sassafras, clove, sage, eucalyptus, marjoram, cinnamon, lemon, lime, grapefruit, orange, apple, pear, peach, strawberry, cherry, apricot, watermelon, banana, coffee, cocoa, menthol, carvone, anethole and combinations thereof.
